(19) United States
(12) Patent Application Publication
(10) Pub. No.: US 2010/0121105 A1
(43) Pub. Date: May 13, 2010

(54) Composition and Method of synthesizing a biomolecule and its therapeutics applications

(76) Inventor: Mehdi Bakhtiari, Karaj (IR)

Correspondence Address: CHOBIN & CHOBIN CONSULTANCY L.L.C, Green Community, Building 3, Ground Floor, Dubai Investment Park, P.O Box 212880, Dubai, 1563714311, IR

(21) Appl. No.: 12/690,126

(22) Filed: Jan. 20, 2010

Related U.S. Application Data
(60) Provisional application No. 61/254,922, filed on Oct. 26, 2009.

(51) Int. Cl. C07C 249/00

(52) U.S. Cl. 562/560

(57) ABSTRACT

It is disclosed a method which relates to the ex-vivo of synthesizing J-Factor and its therapeutic application. In-vivo mechanism involves the secretion of Growth Hormone (GH), and its binding to the GH receptor. During enzymatic interaction between GH and the receptor, at least one of its amino acid specifically L-arginine is changed to J-factor by the bond shifting action in the guanidino group of L-arginine. Finally the GH is broken down into its respective amino acid units within the cell, and the J-Factor starts to accumulate gradually. Thus accumulated J-factor stimulates the synthesis and secretion of autocrine-paracrine IGF-1. IGF-I induces changes in the cell which prevent aging signs. The method further relates to ex-vivo synthesis of J-factor which is responsible for the changes in the cell and its administration to humans and animal. In one of the preferred embodiment the ex-vivo synthesized J-factor is administrated to the patients and the changes in the patients was observed.

### CLAIM PRIORITY

This patent application claims priority to U.S. Provisional Patent Application Ser. No. 61/254,922, filed Oct. 26, 2009, which is incorporated by reference in its entirety.

### FIELD OF INVENTION

In general present invention relates to the field of physiology. While, in particular it relates to ex-vivo synthesis of a biomolecule called J-Factor and its medical application.

### BACKGROUND OF THE INVENTION

Most aging individuals die of various health related issues; but during old age loss of muscle strength and weakening of bone results in frailty, thus limiting an individual's chances of living an independent life until death. Physical changes during aging have been considered physiologic, but there is evidence that some of these changes are related to decline in hormonal activity. Science recognizes aging as a disease that can be reversed to a large degree by increasing GH (Growth Hormone) levels. Biological aging is closely associated with a decline in the capacity for protein synthesis which has been hypothesized to contribute to the decline in tissue function and increased susceptibility to disease. GH and Insulin-Like Growth Factor-1(IGF1) are important anabolic hormones that regulate metabolic processes such as protein synthesis in almost all tissues throughout lifespan. GH is also required for normal postnatal growth, having a critical role in bone growth as well as important regulatory effects on protein, carbohydrate, and lipid metabolism. The physiological effects of GH are brought about by the GH receptor.

### DESCRIPTION OF THE INVENTION

The present invention is related to ex-vivo synthesis of a biomolecule found in-vivo and involved in the signaling and stimulation of autocrine-paracrine Insulin like Growth Factor-I (IGF-I) secretion in different tissues in the body.

It was noticed that any kind of exercises such as jogging, running, bicycling, walking or skating reduced the body fat mass and increased the lean muscle mass. During the exercise, blood glucose is utilized by the body cells leading to the decrease in plasma/blood glucose, which in turn induced the growth hormone (GH) production thereby stimulating autocrine-paracrine IGF-1 signaling leading to the decrease in fat tissue by lipolysis, and strengthening and development of muscles-myogenesis. These changes effectively occurred after a delay from the time when the exercise is started and continued. In addition, there is a decrease in these changes after a delay from the time when the exercise is stopped.

In other words, during the course of the exercise, the above changes were not a gradual process from the beginning. This is because ‘the factor’ responsible for inducing the changes at first accumulates in the cell. Thus the accumulated factor stimulates the production of autocrine-paracine IGF-1.

The Growth hormone harbors the above said factor which is responsible for production of autocrine-paracine IGF-1. The Growth hormone is broken down into its respective amino acids within the cell after interaction with its receptor, wherein said factor is formed from one of the amino acids of GH, which begins to accumulate in the cell, and then stimulating the secretion of IGF-I.

It is noticed that in a premature neonates with very low birth weight (VLBW), when fed with dried milk containing casein, are infected with late metabolic acidosis. In this condition, in spite of feeding milk, weight gain and increase in fatty tissue does not happen due to the increase in urea. This indicates that urea causes the decrease in fatty tissue.

If we consider that stimulating the secretion of autocrine-paracrine IGF-1 by urea, as the reason for the decrease in fatty tissue, the ‘Factor’ stimulating the secretion of IGF-1 should have the following moiety in its chemical structure.

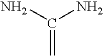

Urea has a chemical formula:

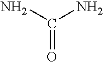

Since, said ‘Factor’ is one of the amino acids of GH, next step of the present invention is to examine the amino acid of GH having the following moiety in its chemical structure.

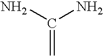

It was found that none of the amino acids of extracellular GH before joining its receptor on the cell surface had said above said shown moiety, but it was observed that if the double bond in the guanidino group (NH2-C(═NH)—NH) of L-arginine which is one of the amino acids of extracellular GH changes its place, then new bio-molecule having the following moiety

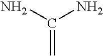

in its chemical structure is possible.

This new biomolecule having the above moiety in its structure is referred for this invention as L-2-amino-5-diaminomethyleneamino-pentanoic acid or ‘J-Factor’. The below diagram shows the transition of the double bond in L-arginine to J-factor:

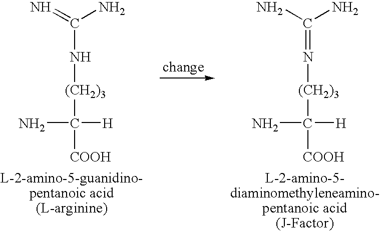

Therefore it's reasonable that at least one of the L-arginines existing in the structure of extracellular GH changes into J-Factor to be able to stimulate the secretion of IGF1.

Extracellular GH does not have J-Factor in its structure. Consequently, the change should be done after GH joins its receptor and before it's broken down into its amino acids in the cells. If the L-arginine changed after GH was broken down, the other L-arginine biomolecules not existing in GH should be considered. Accordingly the receptor of GH changes at least one of the L-arginines of GH into J-Factor by enzymatic action and finally, after GH is broken down into its amino acids, J-Factor accumulates in the cells and stimulates autocrine-paracrine IGF-1.

In one of the embodiment of the present invention L-arginine was chemically converted into J-Factor ex-vivo. Thus converted/synthetic produced ex-vivo J-Factor was orally administrated to a small group of 11 volunteers. Each volunteers were given 0.5 mg per 10 days (=1.5 mg/month) and photographs were taken in each case at the beginning and end of the trial to record the changes in their faces and heads.

The following physical changes in were observed in case of each voluntary:
Hair growth;
Muscular hypertrophy;
A decrease in fatty tissue e.g. in abdomen;
Improvement in eye's central vision; and
Improved tone and color of facial skin;

These changes indicate that ex-vivo synthesized J-Factor functioned like GH and autocrine-paracrine IGF-1 and also stimulated IGF-1 production.

The mechanism that takes place after GH interacts with its receptor is, at least one of its L-arginines is changed to J-Factor by the enzymatic interaction with the receptor. GH is broken down into its respective amino acid units within the cell, thus allowing accumulation of J-Factor gradually and to stimulate the secretion of autocrine-paracrine IGF-I.

### Example 1

### Use of the Invention or Synthetic J-Factor

According to the above theory, after oral administration synthetic J-Factor accumulates in the body's different cells and stimulates the synthesis and secretion of autocrine-paracrine IGF-1.

Consequently, it could have anti-aging effect and the following uses that are the same as the autocrine-paracrine IGF-1 functions in the body which includes but not limited to:
Muscular hypertrophy and restoring the muscle mass, thus giving a toned up and youthful look;
Increase in bone density, to avoid bone fracture which is common in old people; and
Decrease in fatty tissue, to avoid obesity-related diseases such as diabetic, cardiovascular problems etc.
Improvement in eye's central vision;
Hair growth;
Decrease in cellular proptosis;
Improvement in skin elasticity and
other IGF-1 effects.

In fact all of the above mentioned changes help in preventing or limiting the aging signs. The advantage of using ex-vivo synthesized J-Factor of the present invention when compared to biosynthetic GH is shown in the comparative table below:

| Synthetic J-Factor | Biosynthetic GH |
|---|---|
| Oral administration | Intra muscular (IM) |
| | administration |
| 3 times a month | 3 time a week |
| Non-diabetogenic | Diabetogenic |
| Non-antigenic | Antigenic |
| Easily synthesized | Biosynthetic |
| Synthetic J-Factor of the present invention | Complications: |
| showed no complications such as Joint | Joint swelling Carpal tunnel |
| swelling, Carpal tunnel syndrome or Joint | syndrome and Joint pain |
| pain in the 11 volunteers which are common | |
| while using biosynthetic GH. | |

### Example 2

### Method of Synthesizing the J-Factor Ex-Vivo

For ex-vivo synthesis of J-Factor starting material includes but not limited to amino acid, peptide or polypeptide.

We can synthesize J-Factor according to following reactions as in reaction 1, while reaction 2 show the same reaction of peptide chain having L-arginine and its transition to J-factor.

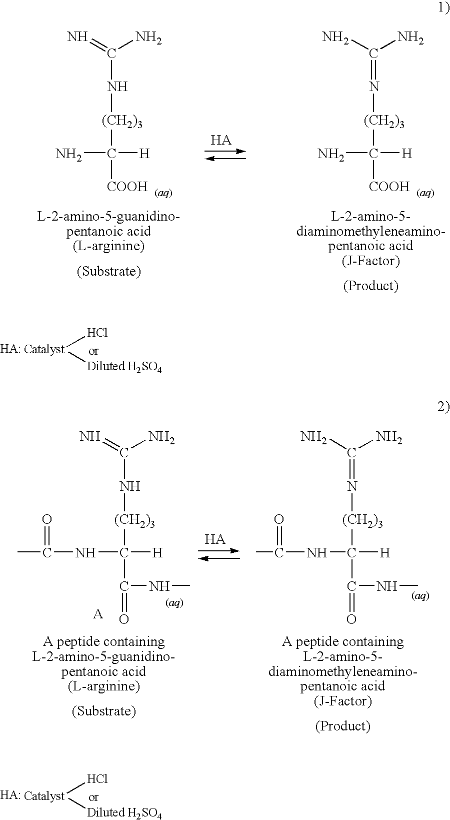

The above reactions are based on the following mechanism in guanidine:

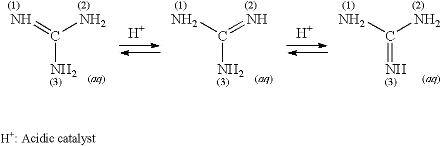

J-Factor synthesis reaction: below reaction illustrators how the L-arginine in presence of an acid catalyst such as hydrochloric acid or diluted sulfuric acid is converted to J-factor in ex-vivo condition in the present invention:

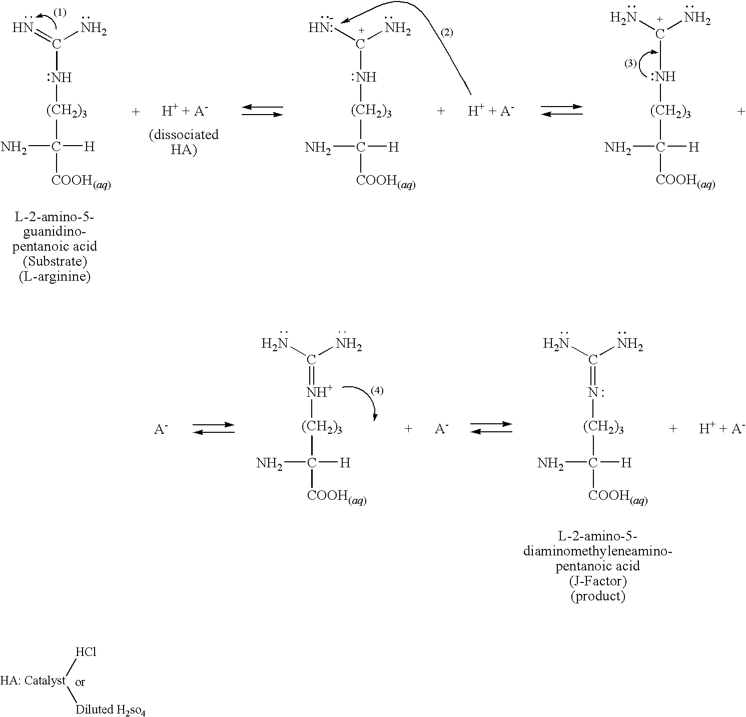

Once the chemical reaction equilibrium is established in the acidic solution and J-Factor concentration becomes maximal, the acidic catalyst (HCl or diluted H₂SO₄) is separated from the solution by
1) anion exchange resin column chromatography:

R=Resin R—OH+HCl→R—Cl+H₂O

2R—OH+H₂SO₄→R₂SO₄+2H₂O

or
2) gradually dissolving Ba(OH)2 into the solution of the diluted H2SO4, while its pH value is constantly measured using a electric pH meter to obtain the pH value 7.0:

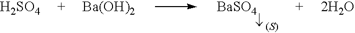

Due to high concentration of H+; hydroxide OH⁻ anions induced by ionization of Ba(OH)2 or R—OH in anion exchange resin column chromatography almost only combines with H+.
Therefore OH⁻ seldom attacks moiety

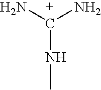

according to the following reactions:

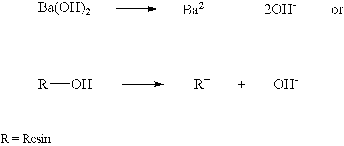

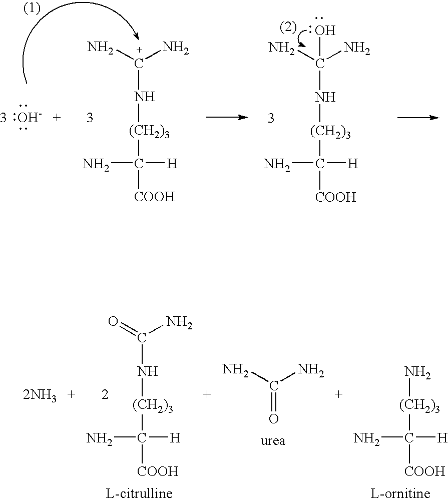

Small amounts of Urea, ammonia (NH₃), L-Citrulline, and L-ornithine are produced as byproducts, when acidic catalyst (HCl or diluted H2SO4) is separated from the solution by the above disclosed methods.
L-Citrulline, L-ornithine are naturally derived from L-arginine in the body and small amounts don't cause any problems. Similarly a small amount of Urea too has no adverse effect on the body. The gaseous byproduct NH₃ is separated from the solution.
On the other hand, after the reaction “L-arginine

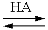

J-Factor” reaches an equilibrium state,

$$\frac{J \text{-} Factor concentration}{L \text{-} arginine concentration} = 0.5 = {K}_{eq}$$

However, it is not required to separate the two bio-molecules because L-arginine is found much more in the daily diet. While you administering J-Factor 50 μg per day, L-arginine is also taken 100 μg per day that is much less than the normal daily dietary requirement the amino acid.

If the J-Factor of the present invention was synthesized from L-arginine or in another route in the body other than from GH and IGF-1 axis; autocrine-paracrine IGF-1 would not decrease along with aging. In addition, the hydrochloric acid in the stomach doesn't practically change L-arginine present in the daily diet to J-Factor as the time is too short.

J-factor in the present invention is formulated into a therapeutic composition in a suitable pharmaceutical acceptable for administering to patients suffering from age-related disorders and diseases.

J-factor of the present invention is prepared for oral administration by mixing J-factor having the desired degree of purity with physiologically acceptable carriers. Such carriers will be nontoxic to recipients at the dosages and concentrations employed.
These therapeutic compositions administered to patients selected from the group consisting of bone-fracture, wound healing, type II diabetic, neurodegenerative conditions, cancer, aging, and muscle wasting diseases.

In another embodiment of the present invention the therapeutic composition for administration is prepared by mixing required concentration of J-factor and L-arginine having desired purity with suitable pharmaceutical acceptable carriers. These therapeutic compositions can further include protein, antioxidants and other such agents which are beneficial and useful to reduce the signs of the aging and age-related disorders.

The invention has been described in connection with its preferred embodiments. However, it is not limited thereto. Changes, variations and modifications to the basic design may be made without departing from the inventive concepts in this invention. In addition, these changes, variations and modifications would be obvious to those skilled in the art having the benefit of the foregoing teachings. All such changes, variations and modifications are intended to be within the scope of the invention which is limited only by the following claims.

What is claimed is:

1. A therapeutic composition of L-2-amino-5-diaminomethyleneamino-pentanoic acid having formula, 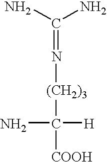
Where N is Nitrogen, O is Oxygen, H is Hydrogen, C is Carbon, ═ is a double bond, — is a single bond, —NH2 represents an amino group, —CH2- represents Methylene, —COOH is a Carboxylic group.

2. The therapeutic composition of claim 1, wherein said formula is derived from l-arginine having formula 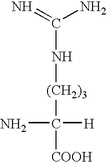
and having a guanidino group,
Where ═NH is an Imine group, and wherein said formula is in presence of an acid as a catalyst, wherein said acid is selected from a group consisting of at least HCl (Hydrochloric acid) and diluted H2SO4 (Sulfuric acid).

3. The therapeutic composition of claim 1, wherein said therapeutic composition is produced by dislocating said double bond in said guanidino group of L-arginine which consist following reaction: 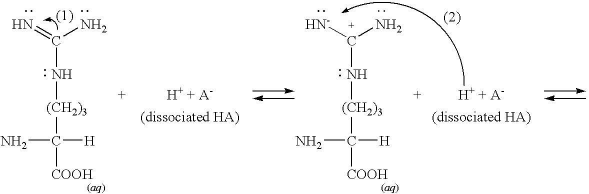
Where HA is acidic catalyst, and is either HCl or diluted H2SO4

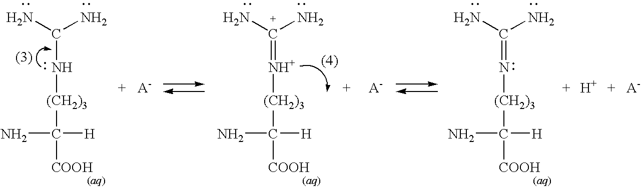

4. The therapeutic composition of claim 1, wherein said therapeutic composition is synthesized from L-arginine which is bounded with at least one amino acid in a peptide chain in presence of an acid as a catalyst, wherein said acid is selected from a group consisting of at least HCl (Hydrochloric acid) and diluted H2SO4 (Sulfuric acid).

5. The therapeutic composition of claim 4, wherein said therapeutic composition is released as a monomer when the bond is broken down in the alimentary canal.

6. A method for making a therapeutic composition which consists of:
moving a double bond in a guanidino group of L-arginine by an acidic catalyst; and
Obtaining said therapeutic composition, wherein said therapeutic composition stimulates the secretion of autocrine-paracrine IGF-1 in tissues in a living body.

7. A method as claimed in claim 6, wherein said therapeutic composition improves hair growth.

8. A method as claimed in claim 6, wherein said therapeutic composition improves muscular hypertrophy and restores the muscle mass, increases bone density, decreases fatty tissue, improves eyes central vision, decreases cellular proptosis, and improves skin elasticity.

9. A method of ex-vivo synthesis of a J-Factor comprises:
performing a chemical conversion of a reactant to a J-factor in presence of an acid catalyst, wherein said reactant is amino acid, peptide, or polypeptide;
performing dislocation of a double bond in said amino acid to form said J-factor;
isolating said J-factor; and
removing said acid catalyst using an anion exchange resin column chromatography.

10. A method according to claim 9, wherein said method further comprises step of: gradually dissolving Ba(OH)2 into a solution of diluted H2SO4, while its pH value is constantly measured using an electric pH meter to obtain a pH value of 7.0.

11. A method according to claim 9, wherein said amino acid have a guanidino group (NH2-C (═NH)—NH) in its chemical structure, further said amino acid having said guanidino group is L-arginine.

12. A method according to claim 9, wherein said peptide and polypeptide have at least one L-arginine in its chain.

13. A method according to claim 9, wherein said acid catalyst is Hydrochloric acid (HCL) or diluted sulfuric acid (H2SO4).
